(19) United States
(12) Patent Application Publication
(10) Pub. No.: US 2018/0103856 A1
(43) Pub. Date: Apr. 19, 2018

(54) AN IMPROVED BLOOD PRESSURE MEASUREMENT SYSTEM

(71) Applicants: UNIVERSITY OF NEWCASTLE UPON TYNE, Newcasttle upon Tyne, Tyne and Wear (GB); A C COSSOR & SON (HOLDINGS) LIMITED, Harlow (GB)

(72) Inventors: Alan MURRAY, Newcastle Upon Tyne, Tyne and Wear (GB); Dingchang ZHENG, Newcastle Upon Tyne, Tyne and Wear (GB); Clive GRIFFITHS, Newcastle Upon Tyne, Tyne and Wear (GB); Chengyu LIU, Newcastle upon Tyne, Tyne and Wear (GB)

(21) Appl. No.: 15/565,896

(22) PCT Filed: Apr. 15, 2016
(86) PCT No.: PCT/GB2016/051065

§ 371 Date: Oct. 11, 2017

(30) Foreign Application Priority Data
Apr. 15, 2015 (GB) 1506420.7

(51) Int. Cl. A61B 5/022; A61B 5/00

(52) U.S. Cl. CPC A61B 5/02225; A61B 5/02208; A61B 5/02233; A61B 2562/0247; A61B 5/7278; A61B 5/742; A61B 5/725

(57) ABSTRACT

A blood pressure measurement system is able to measure actual blood pressures. More specifically, the blood pressure sensor or sphygmomanometer is able to detect high frequencies that exist only between systole and diastole, and thus identify aspects such as micro-pulses associated with the opening and closing of an artery to achieve accurate blood pressure readings.

The present invention relates to an improved blood pressure measurement system which is able to measure actual blood pressures. More specifically the invention relates to a blood pressure monitor or sphygmomanometer which uses a new technique to detect high frequencies that exist only between systole and diastole, and thus identify micro-pulses associated with the opening and closing of an artery to achieve accurate blood pressure readings.

High blood pressure (BP) is one of the leading cardiovascular risk factors for coronary artery disease, congestive heart failure, renal disease and stroke. Despite the importance of BP measurement and its very widespread use, it is generally accepted that it is one of the most poorly performed diagnostic measurements in clinical practice (from the American Heart Association (AHA), and British and European Hypertension Societies (BHS, ESH)) often due to either inadequate operator training or variability between devices.

Sphygmomanometers or blood pressure monitors are well known in the art. Typically they comprise an inflatable cuff, most commonly for positioning around a patient's upper arm at approximately heart level (although in some cases they can be positioned around a patient's wrist or finger), a pressure gauge or transducer for measuring cuff pressure and a mechanism for inflating the cuff to restrict blood flow. There is also a valve to allow controlled deflation of the cuff pressure. In some cases the inflatable cuff can take the form of a pressurisable chamber.

Traditional sphygmomanometers are manual and used by trained practitioners to measure and determine BP of a patient. These devices use a stethoscope for auscultation and require significant training. They must be used in quiet surroundings to allow the practitioner to hear the characteristic sounds, often referred to as Korotkoff sounds. Korotkoff sounds are the audible sounds heard with a stethoscope when it is placed over the brachial artery, distal to the sphygmomanometer's cuff. The sounds are typically heard when the cuff pressure is below systolic blood pressure (SBP) and above diastolic blood pressure (DBP), but suffer from the problem that these sounds can be heard outside this range, especially below DBP. They are associated with the starting and stopping of blood flow in the artery as the pulsatile blood pressure rises above and falls below the cuff pressure. As the cuff pressure is adjusted, over a range encompassing SBP and DBP, the appearance and disappearance of Korotkoff sounds allows SBP and DBP to be estimated. Manual sphygmomanometers of this type are considered to be the “gold standard” measurements providing the most accurate and reproducible readings.

More recently, automated electronic or digital sphygmomanometers have been developed and are commonly used both in doctors' surgeries, hospitals and at home by patients. Automated devices generally use electronic calculation of oscillometric measurements to determine BP rather than auscultation and as such can be used without significant training, unlike manual sphygmomanometers. They can also be used in a greater range of environments as it is not necessary for the environment to be quiet to obtain the reading. Some automated devices have attempted to use a microphone under the distal edge of the cuff to detect and analyse the Korotkoff sounds but accurate placement over the artery has been a problem. Attempts have been made to improve the efficiency of the automated detection method for Korotkoff sounds, but are complex and difficult to implement.

The oscillometric technique utilized by most automated devices is described briefly in this paragraph. The pulsatile pressure produced by the beating heart causes arteries to expand and contract. This effect is exaggerated when the artery is surrounded by a blood pressure cuff at a pressure between approximately SBP and DBP. The change in volume associated with this effect is transmitted through the soft, but incompressible, tissue of the arm to the inner wall of the cuff, making small changes to the volume of the enclosed gas in the bladder of the cuff. By Boyle's Law this superimposes low amplitude pulsatile changes onto the ‘quasi-static’ pressure in the cuff. For cuff pressure above SBP these pulsatile changes are relatively small, then between SBP and DBP rise steadily to a maximum (typically amplitude 1-2 mmHg) and then fall again to below DBP. The changes in amplitude are gradual and so accurate measurements of SBP and DBP are not possible. However arithmetic algorithms have been developed based on statistics which allow BPs to be estimated from the changing pattern of amplitudes. These techniques and the associated algorithms for determining BPs are well known in the art. In some cases the cuff pressures are increased in increments until the required BP readings are obtained, whilst in other cases the cuff is first taken to a high pressure then decreased in increments until the required readings are obtained. It is also possible to provide a smooth rather than incremental pressure reduction.

Although automated digital sphygmomanometers have significant ease of use benefits when compared to “gold standard” manual sphygmomanometers, it has been well documented that there are problems with the accuracy and reproducibility of their blood pressure readings. However, the high skills required to identify Korotkoff sounds using a manual sphygmomanometer has resulted in the popularity of the automated systems using the oscillometric technique, though sometimes an unreliable choice.

It is also known that it is often difficult to detect diastole correctly, even with the manual ‘gold standard’ sphygmomanometers. Traditionally, the systolic blood pressure is taken to be the pressure at which the first Korotkoff sound is first heard and the diastolic blood pressure is the pressure at which the Korotkoff sound is just barely audible; between systolic and diastolic blood pressure, the Korotkoff sounds peak and then begin to fade away. It is however very difficult, even for skilled practitioners, to determine when diastole occurs as the fading away of the Korotkoff sound can be very difficult to judge. In addition, if there is background noise this requires a skilled user with good hearing. The positioning of the stethoscope downstream of the cuff is also important and requires training for correct application. Attempts have been made to position the stethoscope under the cuff itself, at the downstream end of the cuff, in order to minimise movement of the stethoscope during use.

The following paragraphs describe the ‘gold standard’ auscultation method in further details. With the cuff of a sphygmomanometer placed around a patient's upper arm and inflated to a pressure above the patient's systolic blood pressure, and the stethoscope positioned downstream of the cuff over an artery, there is no audible Korotkoff sounds. This is because the pressure in the cuff is high enough such that it completely occludes the blood flow and the artery remains closed.

If the pressure is dropped to a level equal to that of the patient's systolic blood pressure, a first Korotkoff sound should be heard, although small changes in SBP may cause the sound to come and go on successive beats. Repetitive sounds for at least two consecutive beats allow cuff pressure to be considered as the systolic blood pressure. As the pressure in the cuff is allowed to fall further, thumping sounds or murmurs continue to be heard while the pressure in the cuff lies above the systolic and below the diastolic pressures.

Eventually, as the pressure in the cuff drops further, the sounds change in quality, then become muted, and finally disappear altogether. This occurs because, as the pressure in the cuff drops below the diastolic blood pressure, the cuff no longer provides restriction to blood flow allowing the blood flow to become smooth again and thus produce no further audible sound. The disappearance of sound is considered diastolic blood pressure. This “fading away” can be particularly difficult to recognise with it being very difficult to accurately determine when the sounds can no longer be heard.

At present, it is difficult to pinpoint when the actual opening and closing of the artery occurs using any of the current forms of sphygmomanometer.

It is an object of the present invention to obviate or mitigate one or more of the problems associated with both manual and automated sphygmomanometers.

According to a first aspect of the present invention there is a blood pressure measurement system comprising;

a cuff or a chamber which is attachable to apparatus selectively able to pressurise said cuff or chamber;

at least one pressure sensor in fluid communication with the cuff or chamber, said sensor able to sense cuff pressure and variances therein at least above 20 Hz; at least one pressure sensor in fluid communication with the cuff or chamber, said sensor able to sense cuff pressure and variances therein equal to or less than 2 Hz; and

means to analyse the sensed pressure and variances and to determine blood pressure therefrom.

The new technique described here detects the actual opening and closing of the artery during pressure measurement, and so differs substantially from the two techniques described in the art. As it detects the fast high frequency snap arterial action, it may be termed the “arterial snap technique”.

Preferably, said at least one sensor able to sense cuff pressure and variances therein of at least above 20 Hz is capable of sensing the high frequency signals produced by the subtle pressure change associated with the artery opening and closing at systolic and diastolic blood pressure respectively. The baseline cuff pressure may be detected by said at least one sensor able to sense cuff pressure and variances therein at least below 2 Hz. This allows identification of SBP and DBP in conjunction with the high frequency micro-pulses detected by said at least one sensor.

It has been found that when cuff pressure was between SBP and DBP, superimposed on the rising pressure component of the oscillometric waveform a very low amplitude fast negative pressure deflection (amplitude of the order 0.05 mmHg) was observed. This pressure change immediately preceded the Korotkoff sound and was heard over the brachial artery near the elbow. This small micro-pulses disappeared when cuff pressure was above SBP and below DBP.

The origin for the observed micro-signal is believed to be as follows. When cuff pressure is between SBP and DBP, during each heart beat arterial pressure starts to rise. Initially the artery is occluded by the cuff pressure. When arterial pressure reaches cuff pressure, the ‘dam bursts’ or the ‘flood gates open’ and there is a sudden surge of blood through the previously occluded artery. This results in a net small, brief reduction in volume that is detected (by Boyle's law) as the very small amplitude, fast reduction in cuff pressure. The very small amplitude, fast reduction in pressure may in turn be detected by a very high sensitivity, high frequency response pressure sensor. This is immediately overtaken by continuation of the rising pressure of the oscillometric waveform as the previously occluded artery continues to fill.

This waveform may be further discriminated by appropriate signal processing including band pass filtering and because the main components of the signal are in the low audio bandwidth, a conventional sensitive microphone that can continue to function in the presence of high cuff pressure may provide a suitable detector. The waveform disappears above SBP (when the artery remains occluded) and below DBP (when the artery remains open) because there is no sudden surge and so it can provide a basis for accurate measurement of true blood pressures. Further, as the micro-signal can be detected by signal processing and computer algorithms via general principles known in the art, it provides the basis for an automated technique.

As such, the present invention does not rely on detection of Korotkoff sounds or estimates based on oscillometric techniques to determine BP and so obviates or at least mitigates the problems associated with manual and automated sphygmomanometers known in the art. Further, the present invention is non-invasive and relies on the detection of signals associated with the fluid in the cuff, which provides benefits over the auscultation methods described in the art.

Preferably the system includes apparatus for selectively pressurising said cuff or chamber.

Preferably the cuff or chamber is in pneumatical communication with the pressure sensor(s).

Optionally there is a single pressure sensor that is able to sense cuff or chamber pressure and variances therein above 20 Hz and sense baseline cuff or chamber pressure and variances therein below 2 Hz.

The low frequency pressure sensing allows measurement of the baseline cuff or chamber pressure giving a baseline pressure when pressurising/inflating or depressurising/deflating, whilst the higher frequency pressure sensing allows measurement of the snap micro-pulses identified by the inventors. As the system measures both high frequency variances in pressure and baseline cuff pressure it allows determination of micro pulses associated with systole and diastole.

Optionally said pressure sensor is able to sense both cuff or chamber pressure, and variances therein at least up to 100 Hz.

Optionally said pressure sensor is able to sense both cuff or chamber pressure, and variances therein at least up to 200 Hz.

Optionally said pressure sensor is able to sense both cuff or chamber pressure, and variances therein at least up to 300 Hz.

It will be appreciated that in certain embodiments the sensors can read beyond the identified upper frequencies.

Optionally the reading from the pressure sensor is filtered electronically to remove components below 20 Hz or 30 Hz.

Preferably a high pass digital filter is used.

Optionally the filtered signal is enhanced by the multiplication of a transfer function, reducing signals with low amplitude and enhancing the signals with large amplitude.

A classic band-pass analogue/digital filter between 20, or 30, and 300 Hz may be used.

Preferably the pressure sensor is part of the cuff or chamber. Preferably the pressure sensor is integrated into a wall of the cuff or chamber.

Alternatively the pressure sensor is in close proximity to, or is proximal to, the cuff or chamber.

Alternatively, the system comprises a first high frequency pressure sensor and a second low frequency pressure sensor.

Preferably the high frequency pressure sensor is a microphone.

Alternatively the high frequency pressure sensor senses at least above 20 Hz, or above, approximately 30 Hz.

Preferably the high frequency pressure sensor senses from approximately 20 Hz to approximately 300 Hz.

Alternatively the high frequency pressure sensor senses from approximately 30 Hz to approximately 300 Hz.

Alternatively the high frequency pressure sensor senses from approximately 20 Hz to approximately 100 Hz.

Alternatively the high frequency pressure sensor senses from approximately 30 Hz to approximately 100 Hz.

It will be appreciated that in certain embodiments the sensors can read beyond the identified upper and lower frequencies.

Preferably the high frequency pressure sensor is part of the cuff or chamber. Preferably the high frequency pressure sensor is integrated into a wall of the cuff or chamber.

Alternatively the high frequency pressure sensor is in close proximity to, or is proximal to, the cuff or chamber.

Preferably, the second low frequency pressure sensor is able to sense baseline cuff or chamber pressure and variances therein at least below, or below, 2 Hz.

Optionally the reading from the high frequency pressure sensor is filtered electronically to remove components below 20 Hz.

A classic band-pass analogue/digital filter between 20 and 300 Hz may be used.

Optionally the reading from the high frequency pressure sensor is filtered electronically to remove components below 30 Hz.

A classic band-pass analogue/digital filter between 30 and 300 Hz may be used.

Preferably the low frequency pressure sensor is part of the cuff or chamber. Preferably the high frequency pressure sensor is integrated into a wall of the cuff or chamber.

Alternatively the low frequency pressure sensor is in close proximity to, or is proximal to, the cuff or chamber.

Optionally the system comprises a means for processing the information sensed by the pressure sensor. This may be any form of processor and may include basic mechanical processing and/or electrical processing.

The means for processing may be a microprocessor.

The means for processing may be an analogue processor or a digital processor.

The system may include means for storing the information from the pressure sensor.

Preferably the system comprises a display means for displaying information to a user.

The system may for example display real-time cuff pressure (as for a conventional device), stored ‘micro-pulse’ waveform, systolic blood pressure (SBP) and diastolic blood pressure (DBP), heart rate and/or heart rhythm.

Preferably, the filtered signal is processed by the multiplication of a transfer function. This process reduces the signals with low amplitude, and enhances the signals with relatively large amplitude.

Optionally, at beat-to-beat level, one segment of high frequency cuff pressure change, which is defined from a fixed timing window referenced to its corresponding foot of the low frequency oscillometric pulse, is further processed within the timing window for noise reduction and for accurately identifying micro-pulse using a low pass filter and the original segment is then replaced by the filtered segment for better BP determination.

Optionally, blood pressure readings can also be determined from the sudden changes of the time difference information between the foot of oscillometric pulse and the peak of high frequency cuff pressure changes.

According to a second aspect of the present invention, there is provided a method for measuring blood pressure comprising:
sensing a first high frequency signal of greater than at least 20 Hz associated with systolic blood pressure;
sensing low frequency signals of equal to or less than at least 2 Hz associated with a baseline pressure;
sensing a second high frequency signal of greater than at least 20 Hz associated with opening of the artery at diastolic blood pressure; and
determining the blood pressure by analysing the first and second high frequency signals and the low frequency signals.

Preferably the high frequency and low frequency signals are detected non-invasively.

In one embodiment, the method further comprises the step of applying pressure to a blood vessel using a pressurizable cuff or chamber to occlude the blood vessel and gradually reducing the pressure applied until the blood vessel reopens. Preferably the pressure applied is reduced until the blood vessel fully reopens.

Preferably, the first and second high frequency signals are sensed by cuff pressure and variances therein. Additionally, the low frequency signals may be sensed by cuff pressure and variances therein.

Preferably, the method further includes the step of sensing all high frequency signals of greater than at least 20 Hz between systolic and diastolic blood pressure. It is appreciated that between systolic and diastolic blood pressure when the artery is constricted, it opens and closes with every beat, creating high frequency signals, preferably sensed by cuff pressure and variances therein.

According to a third aspect of the present invention, there is provided use of the system of the first aspect of the present invention to measure blood pressure by sensing high frequency signals associated with systolic blood pressure and diastolic blood pressure.

The preceding discussion of the background to the invention is intended only to facilitate an understanding of the present invention. It should be appreciated that the material referred to was part of the common general knowledge as at the priority date of the application.

Throughout the description and claims of this specification, the words “comprise” and “contain” and variations of the words, for example “comprising” and “comprises”, means “including but not limited to”, and is not intended to (and does not) exclude other components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Throughout this document, reference to an automated sphygmomanometer relates to both fully automatic devices where a cuff or chamber is pressurised and depressurised e.g. by an electronically operated pump and valve, and to semi-automatic devices where the cuff or the chamber is inflated or pressurised by hand using a pumping bulb.

It should be noted that where reference is made to a cuff or an inflatable cuff a skilled person would understand that this could be a chamber or pressurisable chamber that itself may have no inner layer.

Features, integers or characteristics, and compounds described in conjunction with a particular aspect, embodiment or example of the invention are to be understood to be applicable to any other aspect, embodiment or example described herein unless incompatible therewith.

There now follows a description of a preferred embodiment(s) of the invention, by way of non-limiting example, with reference being made to the accompanying drawings, in which:

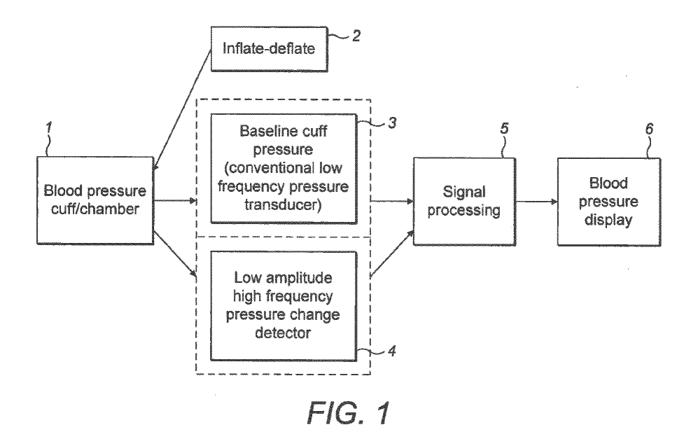

FIG. 1 is a diagram showing a generic system layout for the system of the present invention;

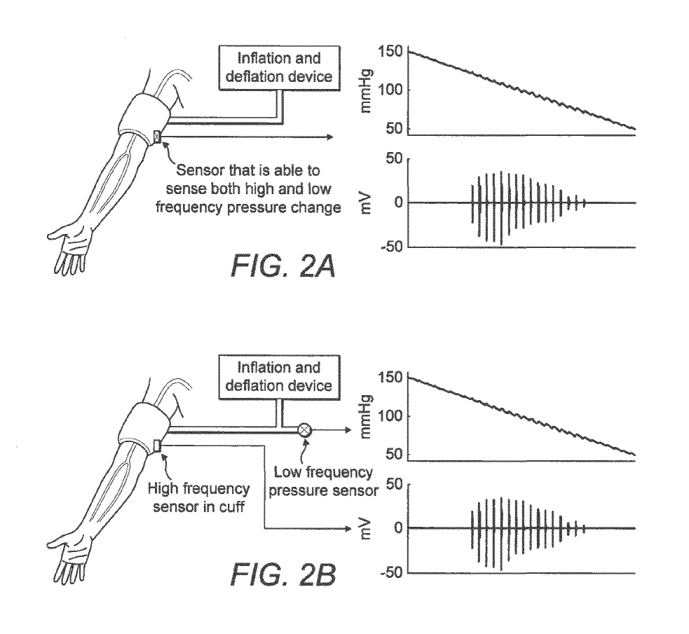

FIG. 2 (A and B): Examples of blood pressure measurement system set ups (left), and example of cuff pressure waveforms (top right of each window) and high frequency cuff pressure changes (right bottom of each window) recorded from the blood pressure cuff;

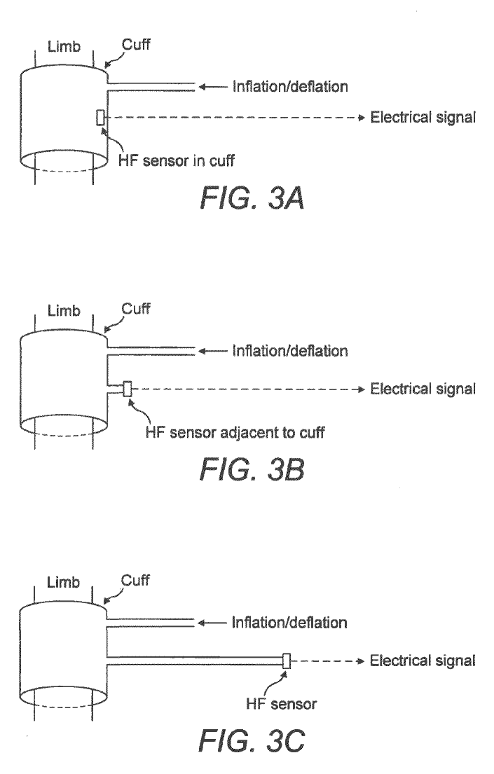

FIG. 3: Three possible embodiments of high frequency (HF) pressure sensor location.

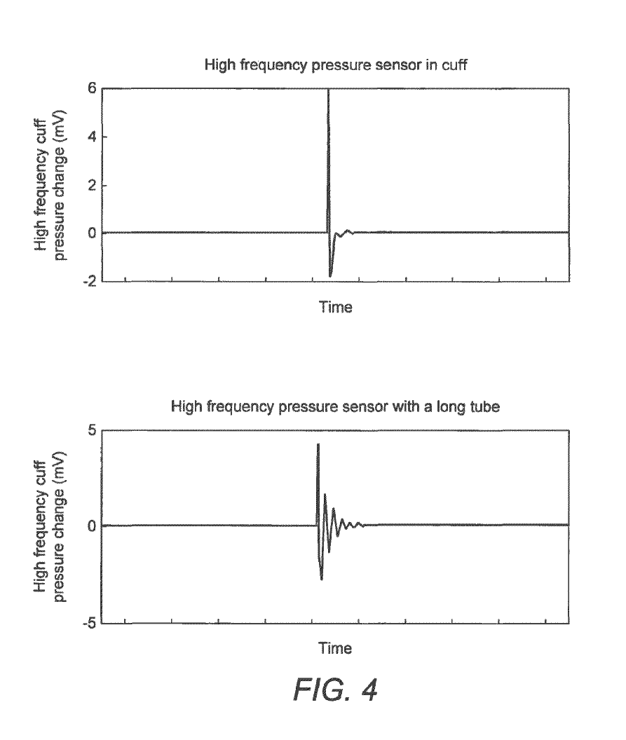

FIG. 4: High frequency cuff pressure changes recorded with the sensor at two locations.

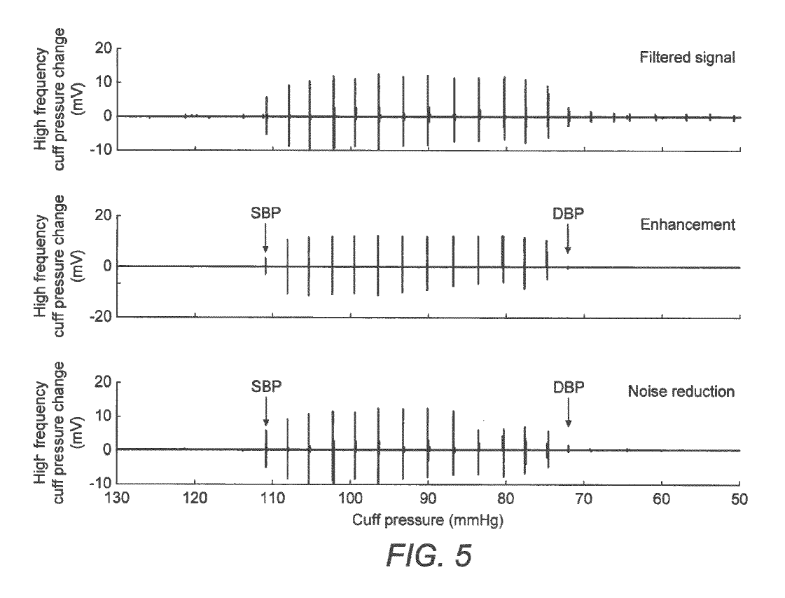

FIG. 5: Processed high frequency cuff pressure changes. Top: Filtered high frequency cuff pressure changes using 30-300 Hz band pass filter; Middle: with further processing of noise reduction (which may be required if noise is present; Bottom: with further process of enhancement.

Note: This example clearly shows that more accurate BP determination could be achieved after further processing, from which the noises above SBP and below DBP are reduced.

Enhancement: The filtered signal is processed by the multiplication of a transfer function. This process reduces the signals with low amplitude, and enhances the signals with relatively large amplitude.

Noise reduction: At beat-by-beat level, one segment of high frequency cuff pressure change, defined from a fixed timing window referenced to its corresponding foot of the low frequency oscillometric pulse, is further processed for noise reduction using a low pass filter to better identify micro-pulses. The original segment is then replaced by the filtered segment.

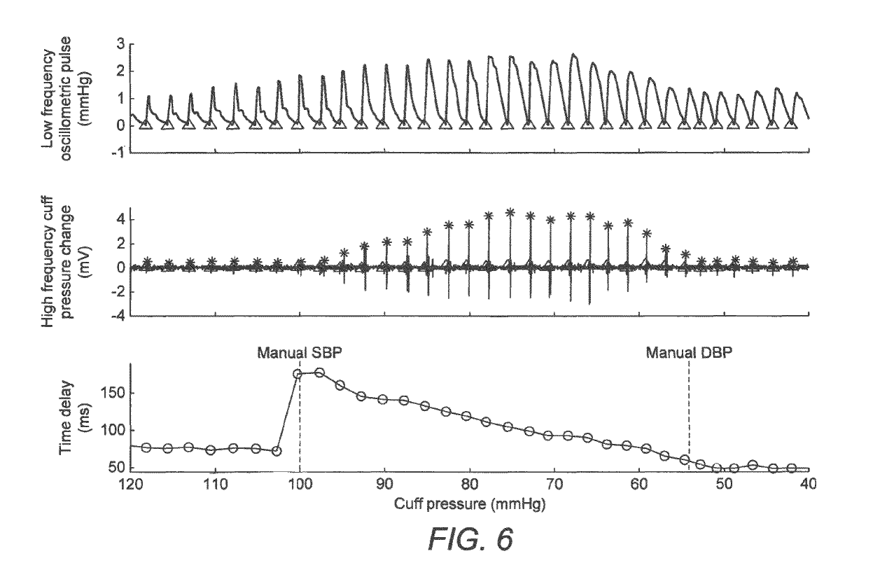

FIG. 6: Low frequency oscillometic pulses (top), and cuff pressure changes (middle) that include components of both the high frequency micro-signal pulses and the oscillometric waveform, enabling the position of the arterial opening pulses relative to the oscillometric pulses to be more easily seen. On the bottom part of the figure, the time difference between the foot of the oscillometic pulses (marked with Δ) and the peaks of the cuff pressure changes signal (marked with *) are shown. Initially these times simply detect the leading edge of the oscillometric pulses, but as soon as SBP is reached the timing suddenly changes to the peak of the oscillometric pulse, and then continues to shorten until DBP is reached. Analysis of the high frequency snap micro-signal pulse relative to the oscillometric pulse enables a detection time window to be set, helping to exclude noise and making the detection of SBP and DBP more accurate in noisy conditions.

A general system according to the present invention is shown in FIG. 1. The blood pressure cuff or chamber 1 may be a conventional cuff or may be of a custom design including that of a chamber without inner layer (the chamber typically having a lower internal volume c.f. a conventional cuff).

In a preferred embodiment the cuff or chamber incorporates a microphone, however this could instead be in close proximity to the cuff or chamber or in fact remote from the cuff or chamber, for example in a main device body which houses the processor. A low frequency pressure transducer is also present to read baseline cuff pressure. In certain embodiments a single pressure sensor or pressure transducer reads both low frequencies and high frequencies.

The system may include a means of inflating/deflating the cuff 2 or a means of pressurising/depressurising the chamber if that is being used. Cuff inflation/deflation of chamber pressurisation/depressurisation may be either manual or automated as is known from conventional sphygmomanometer technology. Alternatively such means 2 may not form part of the system but the system may be attachable to said means.

The system reads both baseline cuff pressure 3 and low amplitude high frequency pressure changes 4.

The system may comprise additional signal processing 5 utilising hardware and/or software (which optionally may be included in the device body). This may include one or more of; a microphone amplifier, an analogue filter, a digital filter, digital discrimination, noise reduction means, micro pulse enhancement means, and pulse detection algorithms. Signal processing and enhancement can provide more accurate BP determination as “noises” above SBP and below DBP are reduced.

Finally, the recorded, and potentially further processed readings are displayed 6 to a user. Typically this will be via a visual display integrated with the processer, for example in a device body. The information could however be sent, using known data transfer technology, to a remote device such as a computer, laptop, mobile device for display or manipulation. As well as BP readings the following information could be obtained or determined and displayed; real-time cuff pressure (as for conventional device), stored ‘micro pulse’ waveform, SBP and DPB, heart rate and heart rhythm.

Determination of SBP and DBP can be carried out by;

manually positioning cursors on the displayed waveform trace of the micro pulse;

automatic detection of SBP and DBP based on:
the appearance and disappearance of processed micro-pulses, or
time difference between initial hydraulic pulse (or oscillometric pulse foot) and micro- pulse
   automatic detection with manual override.

Using the system of the present invention, auscultation with a stethoscope is no longer required as accurate BP readings can be obtained based on the micro-pulses detected. Examples of the blood pressure measurement system set up alongside recorded waveforms are shown in FIG. 2. FIG. 2A shows a system where a cuff has a single pressure transducer or pressure sensor integrated into the wall of the cuff that is able to sense and record both low frequency (at least 0-2 Hz) and high frequency (at least greater than >20 Hz) pressure variances. FIG. 2B shows a system where a cuff has a first high pressure transducer or pressure sensor integrated into the wall of the cuff that is able to sense and record high frequency (at least greater than >20 Hz) pressure variances and a low frequency pressure transducer or pressure sensor that is able to sense and record low frequency (at least 0-2 Hz) remote from the cuff but in pneumatic communication therewith (for example in tubing associated with both the cuff and a means for inflating the same). In both 2A and 2B example cuff pressure waveforms are shown in the top right and example high frequency cuff pressure changes (micro-pulses) are shown in the bottom right.

The system directly senses the pressure variances in the air or fluid in the cuff or chamber (or “listens” to the sounds caused by changes in air or fluid pressure in the cuff). As the cuff now both acts as the site of restricted/released blood flow and the site of sensing pressure variances it is hypothesised that this allows the opening and closing of the artery to be more clearly sensed, in turn allowing systolic and diastolic blood pressure to be determined using the small micropulses that have been identified by the inventors using their direct readings. The system can therefore directly detect those pressure changes resulting from the artery opening and closing below the cuff, termed micropulses herein, and is not reliant on the arterial pulse producing an oscillometric pressure waveform (from which the blood pressures can only be estimated).

In use, a cuff (which could be replaced with an appropriate chamber) is placed around an individual's arm at approximately heart height. In one embodiment, the cuff comprises a pressure sensor in the form of a sensor located on the wall of the cuff. FIG. 3 shows three further possible variants where a high frequency transducer or pressure sensor is located at different positions, however it would be understood by one skilled in the art that a single pressure sensor able to sense high and low frequencies could equally by placed in such positions.

In a preferred embodiment the sensor is embedded into the wall of the cuff and heat sealed in place. The cuff is inflated to a pressure in excess of systolic pressure; this may be manually or under the control of a processor. Optionally, pulse wave amplitude value, heart rate, systolic blood pressure and various other parameters can be sensed and recorded during inflation of the cuff if so desired using standard methodologies such as the oscillometric method. It has been found that this particular embodiment where the high frequency pressure sensor (or combined high and low frequency pressure sensor) is integrated into the cuff (or chamber) provides a particularly clear signal. FIG. 4 shows high frequency cuff pressure changes recorded with the sensor at two locations. When the sensor is remote from the cuff a resonant effect is observed whereas when the high frequency pressure sensor is in the cuff the resonant effect is not observed.

In certain embodiments, the reading(s) from the pressure sensor or sensors are further processed to give more accurate readings. FIG. 5 shows how processing the variations in high frequency cuff pressure can provide very accurate BP determination. For example, the top graph shows filtered high frequency cuff pressure changes using 30-300 Hz band pass filter. The middle graph shows the effect of further process of enhancement—The filtered signal is processed by the multiplication of a transfer function. This process reduces the signals with low amplitude, and enhances the signals with relatively large amplitude. The bottom graph show further processing with noise reduction—at beat-by-beat level, one segment of high frequency cuff pressure change, which is defined from a fixed timing window referenced to its corresponding foot of the low frequency oscillometric pulse, is further processed for noise reduction. The original segment is then replaced by the filtered segment. These graphs clearly show that more accurate BP determination can be achieved after further processing, from which the noises above SBP and below DBP are reduced.

FIG. 6 shows how the signals can be used to determine BPs accurately using low frequency oscillometic pulses (top) and cuff pressure changes (middle) recorded from the cuff that include components of both the high frequency snap micro-signal pulses and the oscillometric waveform, enabling the position of the arterial opening pulses relative to the oscillometric pulses to be more easily seen.

In summary the present invention allows for accurate determination of true blood pressures. It will be understood that a preferred embodiment has at least the high frequency sensor, transducer incorporated into the wall of the cuff or chamber that is in contact with a person's arm in use—this provides particularly accurate readings. Further, the processing of the signal allows for very accurate results to be obtained with minimal requirements for user training.

1. A blood pressure measurement system comprising:
a cuff or a chamber which is attachable to apparatus selectively able to pressurize said cuff or chamber;
at least one pressure sensor in fluid communication with the cuff or chamber, said sensor able to sense cuff pressure and variances therein at least above 20 Hz;
at least one pressure sensor in fluid communication with the cuff or chamber, said sensor able to sense cuff pressure and variances therein at least equal to or below 2 Hz; and
a computer configured to analyze the sensed pressure and variances and to determine blood pressure therefrom.

2-3. (canceled)

4. A blood pressure measurement system as in claim 1 wherein there is a single pressure sensor that is able to sense cuff or chamber pressure and variances therein above 20 Hz and sense cuff or chamber pressure and variances therein below 2 Hz.

5-6. (canceled)

7. A blood pressure measurement system as in claim 1 wherein said pressure sensor is able to sense cuff or chamber pressure, and variances therein at least up to 300 Hz.

8. A blood pressure measurement system as in claim 1 wherein the reading from the pressure sensor is filtered electronically to remove components below 20 Hz or 30 Hz.

9. (canceled)

10. A blood pressure measurement system as in claim 1 wherein the pressure sensor is part of the cuff or chamber, or is in close proximity to, or is proximal to, the cuff or chamber.

11-12. (canceled)

13. A blood pressure measurement system as in claim 1 wherein, the system comprises a first high frequency pressure sensor and a second low frequency pressure sensor.

14. (canceled)

15. A blood pressure measurement system as in claim 13 wherein the high frequency pressure sensor senses at least above 20 Hz, or above, approximately 30 Hz.

16. A blood pressure measurement system as in claim 13 wherein the high frequency pressure sensor senses from approximately 20 Hz or approximately 30 Hz to approximately 300 Hz.

17-19. (canceled)

20. A blood pressure measurement system as in claim 13 wherein the high frequency pressure sensor is part of the cuff or chamber, or is in close proximity to, or is proximal to, the cuff or chamber.

21-22. (canceled)

23. A blood pressure measurement system as in claim 13 wherein, the second low frequency pressure sensor is able to sense cuff or chamber pressure and variances therein at least below 2 Hz.

24-25. (canceled)

26. A blood pressure measurement system as in claim 13 comprising a classic band-pass analogue/digital filter wherein the filtered signal is enhanced by the multiplication of a transfer function, reducing signals with low amplitude and enhancing the signals with large amplitude.

27. A blood pressure measurement system as in claim 13 wherein the reading from the high frequency pressure sensor is filtered electronically to remove components below 30 Hz.

28. (canceled)

29. A blood pressure measurement system as in claim 13 wherein the low frequency pressure sensor is part of the cuff or chamber, or is in close proximity to, or is proximal to, the cuff or chamber.

30-31. (canceled)

32. A blood pressure measurement system as in claim 1 wherein the computer is further configured to process the information sensed by the pressure sensor.

33-37. (canceled)

38. A blood pressure measurement system as in claim 1 wherein a segment of high frequency cuff pressure change, which is defined from a fixed timing window referenced to its corresponding foot of a low frequency oscillometric pulse, is further processed using a low pass filter and the original segment is then replaced by a filtered segment.

39. A blood pressure measurement system as in claim 1 wherein blood pressure readings are determined using time difference information.

40. A method for measuring blood pressure comprising:
sensing a first high frequency signal of greater than at least 20 Hz associated with systolic blood pressure;
sensing low frequency signals of equal to or less than at least 2 Hz associated with a baseline pressure;
sensing a second high frequency signal of greater than at least 20 Hz associated with opening of the artery at diastolic blood pressure; and
determining the blood pressure by analysing the first and second high frequency signals and the low frequency signals.

41. The method of claim 40 wherein the high frequency and low frequency signals are detected non-invasively.

42. The method of claim 40 further comprising the step of sensing all high frequency signals of greater than at least 20 Hz between systolic and diastolic blood pressure.

43. A method for measuring blood pressure in an individual, comprising:
providing the system of claim 1;
placing the cuff or chamber around the individual's arm;
pressurizing said cuff or chamber;
sensing high frequency signals associated with systolic blood pressure from said sensor able to sense cuff pressure and variances therein at least above 20 Hz; and
sensing low frequency signals associated with diastolic blood pressure from said sensor able to sense cuff pressure and variances therein at least equal to or below 2 Hz;
analyzing the sensed high and low frequency signals to determine blood pressure therefrom.
